(19) United States
(12) Patent Application Publication
(10) Pub. No.: US 2007/0129641 A1
(43) Pub. Date: Jun. 7, 2007

(54) Posture estimation at transitions between states

(76) Inventor: Robert J. Sweeney, Woodbury, MN (US)

Correspondence Address: SCHWEGMAN, LUNDBERG, WOESSNER & KLUTH/BSC-CRM, PO BOX 2938, MINNEAPOLIS, MN, 55402, US

(21) Appl. No.: 11/291,479

(22) Filed: Dec. 1, 2005

(51) Int. Cl. A61B 5/04; A61N 1/00

(52) U.S. Cl. 600/513; 607/17; 607/27

(57) ABSTRACT

An implanted device includes a posture sensor configured to produce one or more electrical signals associated with an orientation of the posture sensor relative to a direction of gravity. The device also includes a processor coupled to the posture sensor, the processor being programmed to process the electrical signals from the posture sensor using hysteresis, and to estimate one of a plurality of posture states based on the processed electrical signals.

### TECHNICAL FIELD

Embodiments disclosed herein relate generally to posture sensors.

### BACKGROUND

Posture is an important parameter that can affect many physiologic systems and sensing signals. Posture, if estimated over time, can itself be one indicator of an individual's health condition. Posture can also be used to better interpret other physiological measures that depend upon posture. For example, posture estimates can be used to validate caloric expenditure estimates made based on other physiological measures, as described in U.S. patent application Ser. No. 10/892,937 to Baker, filed on Jul. 16, 2004.

Implanted devices including posture sensors are known. A posture sensor can be used to estimate an individual's current posture (e.g., upright, sitting, lying down, etc.). As the individual moves from one posture to another, the posture sensor generates signals indicative of the change in posture, and these signals are used to estimate the individual's posture. Artifactual noise associated with the individual's environment (e.g., electrical, vibration, etc.) can affect the posture sensor. Such issues become more pronounced as the posture sensor approaches a transition between postures, making the posture sensor susceptible to providing incomplete or inaccurate posture sensing.

### SUMMARY

Embodiments disclosed herein relate generally to posture sensors.

According to one aspect, an implanted device includes a posture sensor configured to produce one or more electrical signals associated with an orientation of the posture sensor relative to a direction of gravity. The device includes a processor coupled to the posture sensor, the processor being programmed to process the electrical signals from the posture sensor using hysteresis, and to estimate one of a plurality of posture states based on the processed electrical signals.

According to another aspect, an implanted cardiac rhythm management device includes a posture sensor configured to produce one or more electrical signals associated with an orientation of the posture sensor relative to a direction of gravity. The device includes a processor coupled to the posture sensor, the processor being programmed to process the electrical signals from the posture sensor using hysteresis, and to estimate one of a plurality of posture states based on the processed electrical signals. The device also includes a transceiver module programmed to transmit the estimate of the one posture state to an external device.

According to yet another aspect, a method for estimating posture using an implanted device includes: generating one or more signals indicative of an orientation of the device relative to a direction of gravity; processing the signals by defining a transition band about a transition line between posture states of a plurality of posture states; and estimating one of the plurality of posture states based on the processed signals.

### DESCRIPTION OF THE DRAWINGS

FIG. 1 is a schematic view of an example cardiac rhythm management device associated with a heart.

FIG. 2 is an example method for estimating posture using hysteresis.

FIG. 3 is an example diagram illustrating multiple posture states and a signal from a one-dimensional posture sensor.

FIG. 4 is another example diagram illustrating multiple posture states and a signal from a two-dimensional posture sensor.

FIG. 5 is another example diagram illustrating multiple posture states and a signal from a three-dimensional posture sensor.

FIG. 6 is another example diagram illustrating a discrete-value signal from a posture sensor.

FIG. 7 is another example diagram illustrating a discrete-value signal from a posture sensor.

### DETAILED DESCRIPTION

Embodiments disclosed herein relate generally to posture sensors. For example, example systems and methods disclosed herein relate to the estimation of posture, particularly at the transition between two or more postures. While the disclosure is not so limited, an appreciation of the various aspects of the disclosure will be gained through a discussion of the examples provided below.

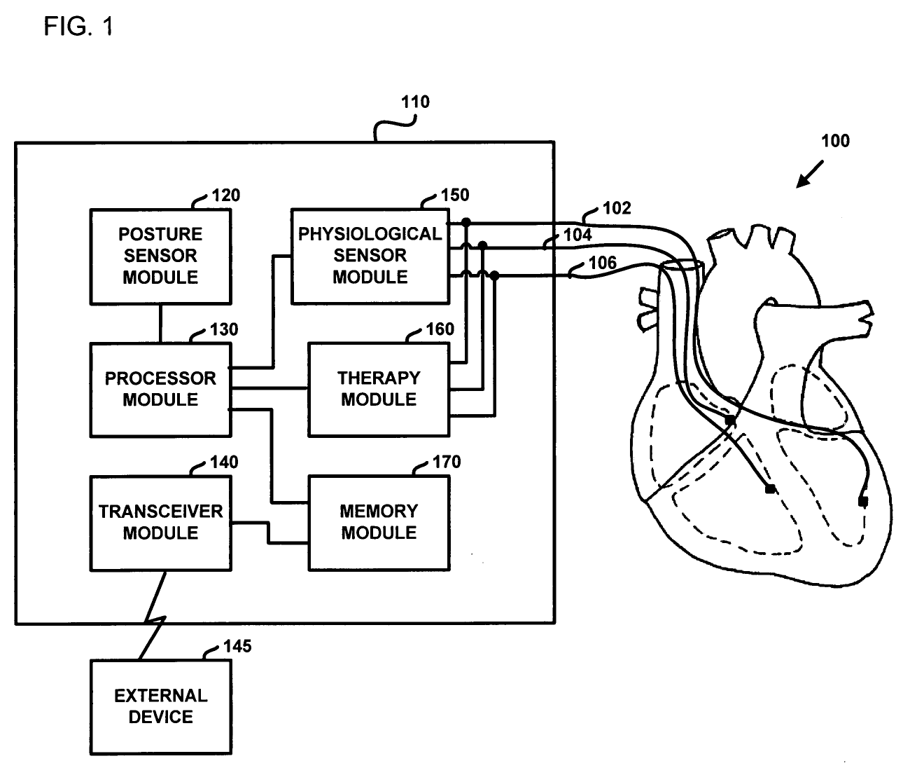

Referring now to FIG. 1, a schematic representation of an example implanted cardiac rhythm management (“CRM”) device 110 is provided. The example device 110 has a plurality of logic units or modules, including a posture sensor module 120, a processor module 130, a transceiver module 140, a physiological sensor module 150, a therapy module 160, and a memory module 170. The device 110 is associated with an individual's heart 100 through leads 102, 104, and 106.

The posture sensor module 120 is used to sense an individual's posture. For example, posture sensor module 120 is configured to sense movement, such as changes in the orientation of posture sensor module 120 relative to the direction of gravity. Posture sensor module 120 is also configured to provide one or more signals indicative of the changes in orientation.

The signal from posture sensor module 120 is used to estimate the individual's posture. For example, the signal can be used to estimate one of a plurality of posture states defining different postures, such as lying, sitting, standing, running, etc. Other states are possible. As an individual moves, the individual can change postures. As the individual changes postures, the orientation of posture sensor module 120 also changes with the individual, and posture sensor module 120 can sense the movement (i.e., change in orientation) and generate a signal indicative of the change. The estimate of the individual's posture state can, in turn, be based on the signal from the posture sensor module 120. For example, if an individual stands up from a sitting position, posture sensor module 120 can sense the change in orientation, and the estimation of posture state can be changed based on the signal from posture sensor module 120.

There are several devices and methods that can be used to sense movement associated with an individual's posture. For example, U.S. Pat. No. 6,658,292 to Kroll et al., the entirety of which is hereby incorporated, discloses a three-dimensional accelerometer that can be used to sense changes in an individual's posture. In another example, U.S. Pat. No. 5,354,317 to Alt, the entirety of which is hereby incorporated, discloses a mechanoelectrical transducer including a suspended plate structure responsive to the earth's gravitational field that can be used to sense posture changes. In yet another example, changes in posture can be sensed using devices that provide discrete values, such as one or more switches located at different orientations with discrete on/off signals. Other devices and methods for posture sensor module 120 are possible.

In examples disclosed herein, posture sensor module 120 can provide one-dimensional, two-dimensional, or three-dimensional signals indicative of the orientation of the module and the individual's current posture. In the illustrated examples, posture sensor module 120 is incorporated as part of a CRM device, such as device 110. In other examples, posture sensor module 120 can be implanted separately from other CRM devices. In yet other embodiments, posture sensor module 120 can be included as a component of an external (i.e., non-implanted) device.

In example embodiments, posture sensor module 120 senses an individual's movements (through a change in the orientation of posture sensor module 120), estimates the individual's posture state, and provides a signal indicative of the estimate of the individual's posture state to, for example, processor 130 described below. In other embodiments, posture sensor module 120 senses an individual's movements and provides one or more signals indicative of the movements to processor module 130, and processor module 130 uses these signals to estimate the individual's posture state. In some embodiments, data from posture sensor module 120 is recorded periodically or in real time using, for example, memory module 170 described below.

The processor module 130 controls the functions of device 110. For example, processor module 130 controls the functions of posture sensor module 120. In addition, in some embodiments, processor module 130 can process one or more signals from posture sensor module 120, and estimate one of a plurality of posture states based on the signals.

The transceiver module 140 allows an external device, such as external device 145, to communicate with device 110. For example, external device 145 can be a programmer that communicates with device 110 using telemetry. In addition, external device 145 can be an interrogator/transceiver unit that collects and forwards data from the device 110 to a central host as part of an advanced patient management system. See the example interrogator/transceiver units disclosed in U.S. patent application Ser. No. 10/330,677 to Mazar et al., filed on Dec. 27, 2002, the entirety of which is hereby incorporated by reference.

In some embodiments, data from posture sensor module 120 can be sent by transceiver module 140, periodically or in real time, to external device 145. For example, in some embodiments data indicative of changes in orientation from posture sensor module 120 is sent by transceiver module 140 to external device 145. In other embodiments, data indicative of the individual's posture state is sent. External device 145 can forward the data, periodically or in real time, to a central host as part of an advanced patient management system.

The physiological sensor module 150 senses physiological data associated with the individual. For example and without limitation, physiological sensor module 150 can be an accelerometer and/or a minute ventilation sensor, both of which are used, for example, in adaptive rate pacing.

The therapy module 160 is used to deliver therapy to the individual. For example, therapy module 160 can be configured to deliver pacing therapy, cardiac resynchronization therapy, and/or defibrillation therapy to the individual through one or more of leads 102, 104, 106.

The memory module 170 stores data associated with the device 110. For example, memory module 170 can store physiological data, as well as derived measurements, such as an estimated posture state provided by posture sensor module 120 and/or processor module 130. The data stored in memory module 170 can be accessed, for example, by external device 145.

The modules associated with device 110 are examples only. Additional or different modules can also be provided as part of device 110. In addition, although example device 110 is an implanted device, other embodiments can include devices external to the individual 's body. For example, in some embodiments, posture sensor module 120 can be part of an external (i.e., non-implanted) device.

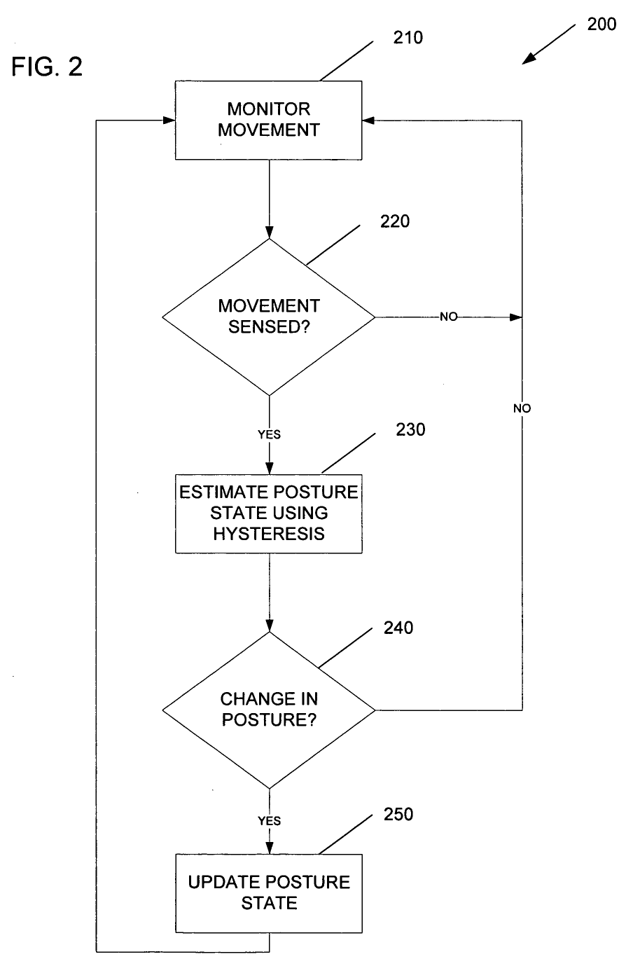

Referring now to FIG. 2, an example method 200 for sensing movement of an individual and transitioning between estimated posture states is shown. At operation 210, movement of the individual is monitored using, for example, a posture sensor. Next, at operation 220, a determination is made regarding whether or not movement is sensed. If no movement is sensed, control is passed back to operation 210 for continued monitoring.

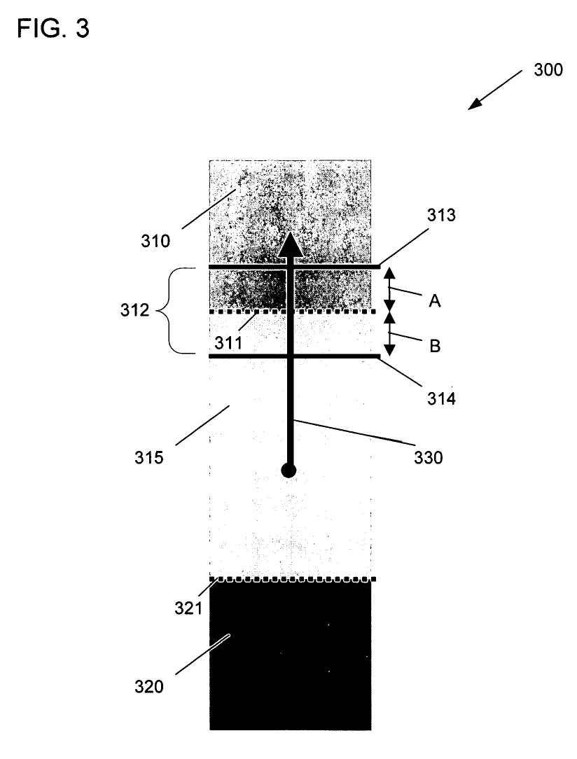

If movement is sensed, control is passed to operation 230, and, in the example embodiment, an estimation of posture state is made using hysteresis. As used herein and described further below, the term “hysteresis” generally means that the current estimated posture state is based not only on the currently-sensed movement of the individual, but also on the previous history of sensed movement. Hysteresis, as described herein, can be expressed as a double-valued function, wherein transitions between posture states are based not on an absolute threshold, but instead include a transition band wherein the estimate of current posture state is based both on the currently-sensed movement of the individual as well as the previous history of sensed movement. See, for example, FIGS. 3-7 described below.

Referring again to FIG. 2, once an estimate of posture state is made using hysteresis, control is passed to operation 240 to determine whether or not a change in posture state has occurred. If a change of posture state has not occurred, control is passed back to operation 210 for continued monitoring.

If a change is posture state has been made, control is passed to operation 250, and the current posture state is updated to reflect the newly estimated posture state. Next, control is passed back to operation 210 for continued monitoring.

Referring now to FIG. 3, an example diagram 300 is shown illustrating three example posture states 310, 315, 320 for a one-dimensional posture sensor. For example and without limitation, in the illustrated embodiment, posture state 310 can be lying down, posture state 315 can be sitting, and posture state 320 can be standing.

A transition line 311 is located between states 310 and 315. In the example shown, a transition band 312 with thresholds 313, 314 is defined about transition line 311. Transition band 312 is used to apply hysteresis to the estimation of the posture state. For example, the estimation of the posture state in transition band 312 is based not only on the currently sensed movement, but also on the previous history of sensed movement.

For example, as illustrated in FIG. 2, the individual's posture is initially estimated to fall within state 315 (e.g., sitting). As the individual moves, example signal 330 represents the amplitude of movement sensed by the one-dimensional posture sensor. As signal 330 approaches and extends into transition band 312, the current posture estimation remains as posture state 315. As signal 330 representing the amplitude of movement extends beyond transition line 311, the current posture estimation continues to be posture state 315 until signal 330 passes threshold 313. After signal 330 exceeds threshold 313, the estimate of posture is updated to posture state 310 (e.g., standing).

Conversely, once the estimate of the posture is at posture state 310, the estimate for posture state will not revert back to state 315 until the amplitude of movement as illustrated by signal 330 passes below transition line 311 and threshold 314.

In example embodiments, interval A between transition line 311 and threshold 313, and interval B between transition line 311 and threshold 314, can be equal or unequal. In some examples, interval A or B is predetermined. In other examples, interval A or B is adapted to an individual based, for example, on the actual variability of the estimated posture states exhibited over time.

In some examples, hysteresis is applied at every transition between estimated posture states, such as at transition line 311, and transition line 321 between state 315 and state 320. In other embodiments, hysteresis is applied only at select transitions, such as, for example, only at transition line 311 as illustrated in FIG. 3.

Transition band 312 can therefore be used to implement hysteresis in the estimation of posture state to reduce changes between states when signal 330 fluctuates around a transition line between posture states.

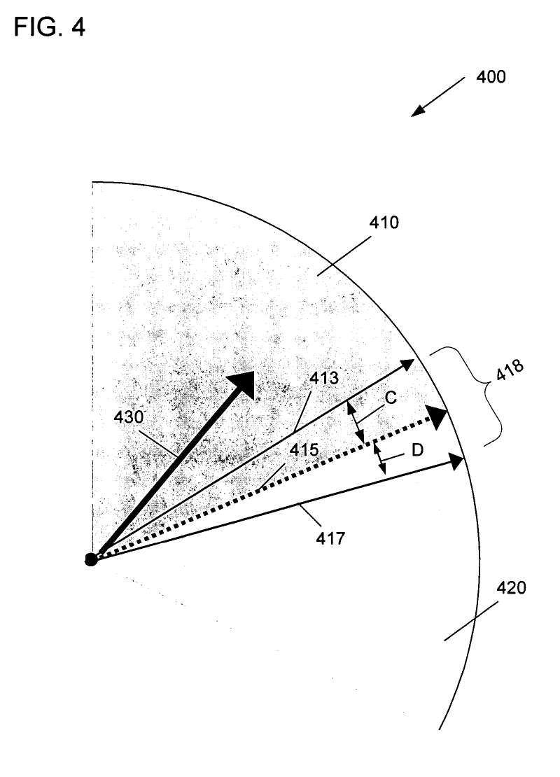

Referring now to FIG. 4, another example diagram 400 illustrating two example posture states 410, 420 for a two-dimensional posture sensor is shown. A transition line 415 is located between states 410 and 420. In addition, a transition band 418 with thresholds 413, 417 is defined about transition line 415.

In the example shown, signal 430 represents the angular direction of movement sensed by the two-dimensional posture sensor. Transition from state 410 to state 420 only occurs if the angular direction of signal 430 passes beyond threshold 417. Likewise, transition from state 420 to state 410 only occurs if the direction of signal 430 passes beyond threshold 413. Angular intervals C and D between transition line 415 and thresholds 413, 417 can be equal or unequal, and can be pre-determined or varied as described above.

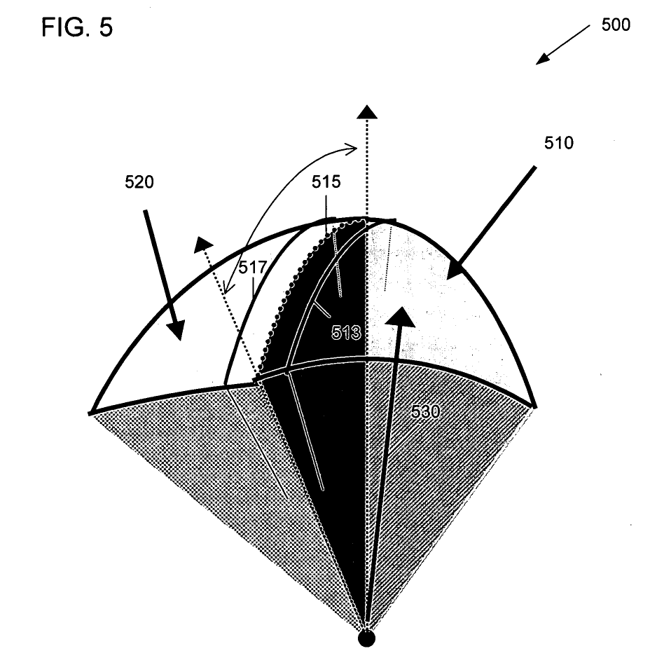

Referring now to FIG. 5, another example diagram 500 illustrating two example posture states 510, 520 for a three-dimensional posture sensor with signal 530 is shown. A transition plane 515 is located between states 510 and 520. In addition, a transition band with thresholds 513, 517 is defined about transition plane 515. Although transition plane 515 and thresholds 513, 517 are illustrated as being linear in the example shown, in other embodiments the transition and thresholds can be non-linear in shape.

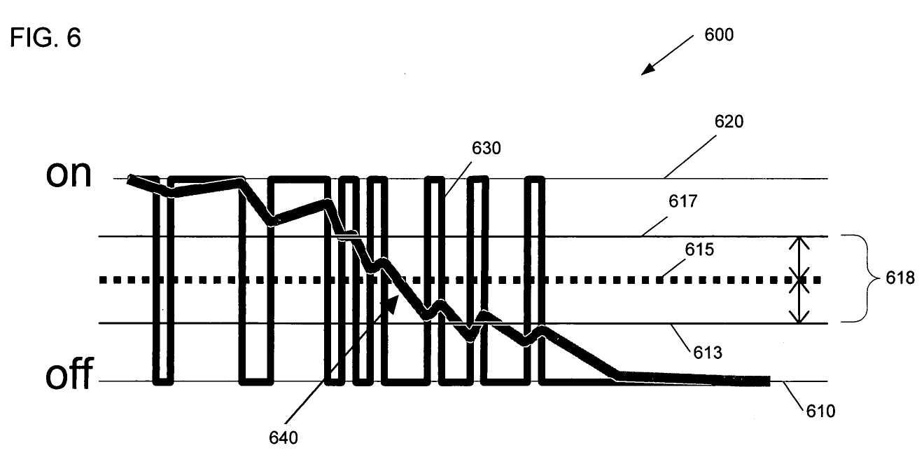

Referring now to FIG. 6, in some embodiments, the posture sensor provides a discrete signal, such as an on/off signal, that can be used to estimate posture. For example, in one embodiment, one or more switches are located at given orientations and provide one or more discrete signals that are used to estimate posture. An example diagram 600 illustrates a discrete signal 630 from a posture sensor. Signal 630 changes over time, as shown on the x-axis of diagram 600, varying between an on state 620 and an off state 610, as shown on the y-axis. A transition line 615 represents the transition from the currently declared posture state to another posture state. In addition, a transition band 618 with thresholds 613, 617 is defined about transition line 615.

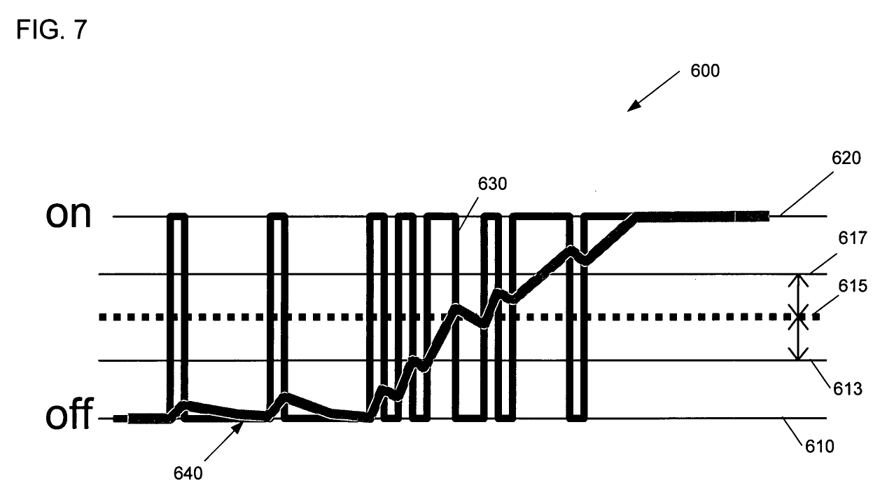

As signal 630 fluctuates between on state 620 and off state 610, a time-average line 640 is calculated. As shown in FIG. 6, line 640 must fall below threshold 613 for the estimate of posture state to be updated from a given state (e.g., state “A”) to a new state (e.g., state “B”). Likewise, as shown in FIG. 7, once the estimate of the posture state is updated to state B, line 640 must exceed threshold 617 before the estimate of posture state is updated back to state A.

As noted above, the thresholds for the transition band between posture states can be varied in size for each transition. In some embodiments, the intervals between thresholds for a given transition band can vary in size. For example, in some embodiments, interval C is greater than interval D as shown in FIG. 4, or vice versa. In other embodiments, one of the two intervals can be eliminated (or logically positioned at the transition line) so that, for example, interval B is eliminated and the estimate for posture state is immediately updated to state 315 when signal 330 falls below transition line 311.

In some embodiments, the thresholds are pre-determined. In other embodiments, the thresholds are tailored for each individual. For example, in some embodiments, the thresholds are adapted to an individual based on the actual variability of the estimated posture states exhibited over time. For example, if the estimated posture state for an individual exhibits a number of fluctuations between two posture states over time, the transition band defined between the two states can be increased in size to minimize the fluctuations.

In some embodiments, multiple posture states can be declared at the same time. For example, instead of maintaining a given estimated posture until the posture signal exceeds a threshold of a transition band, in alternative embodiments two posture states are declared at the same time when the posture signal enters the transition band between the two states. In yet other embodiments, no posture estimate or an indeterminate posture estimate state is provided when the posture signal enters a transition band between two states. Other configurations are possible.

In alternative embodiments, other methods can be used to reduce fluctuations and/or artifactual noise other than hysteresis. For example, in some alternative embodiments, signals of the posture sensor indicative of movement are processed using low-pass filtering techniques to reduce state fluctuations due to, for examples, environmental artifacts (e.g., electrical, vibration, etc.).

Use of the systems and methods disclosed herein to estimate posture at transitions between posture states can exhibit one or more of the following advantages. For example, use of the systems and methods disclosed herein, such as hysteresis, can decrease fluctuation between posture states and thereby provide a more stable estimate of posture state over time. In addition, the susceptibility of posture state estimation to external factors, such as environmental artifacts, can be reduced.

The systems and methods of the present disclosure can be implemented using a system as shown in the various figures disclosed herein including various devices and/or programmers, including implantable or external devices. Accordingly, the methods of the present disclosure can be implemented: (1) as a sequence of computer implemented steps running on the system; and (2) as interconnected modules within the system. The implementation is a matter of choice dependent on the performance requirements of the system implementing the method of the present disclosure and the components selected by or utilized by the users of the method. Accordingly, the logical operations making up the embodiments of the methods of the present disclosure described herein can be referred to variously as operations, steps, or modules. One of ordinary skill in the art will note that the operations, steps, and modules can be implemented in software, in firmware, in special purpose digital logic, analog circuits, and any combination thereof without deviating from the spirit and scope of the present disclosure.

The above specification, examples and data provide a complete description of the manufacture and use of example embodiments disclosed herein. Since many embodiments can be made without departing from the spirit and scope of the disclosure, the invention resides in the claims hereinafter appended.

What is claimed is:

1. An implanted device, comprising:
a posture sensor configured to produce one or more electrical signals associated with an orientation of the posture sensor relative to a direction of gravity; and
a processor coupled to the posture sensor, the processor being programmed to process the electrical signals from the posture sensor using hysteresis, and to estimate one of a plurality of posture states based on the processed electrical signals.

2. The device of claim 1, wherein the processor is programmed to define a transition band about a transition line between two posture states to implement hysteresis.

3. The device of claim 2, wherein the transition band includes first and second thresholds, wherein the first and second thresholds are pre-determined.

4. The device of claim 2, wherein the transition band includes first and second thresholds, wherein the first and second thresholds are varied over time.

5. The device of claim 1, wherein the device is a cardiac rhythm management device.

6. The device of claim 1, wherein the posture sensor is configured to sense the orientation in one dimension.

7. The device of claim 1, wherein the posture sensor is configured to sense the orientation in two or more dimensions.

8. An implanted cardiac rhythm management device, comprising:
a posture sensor configured to produce one or more electrical signals associated with an orientation of the posture sensor relative to a direction of gravity;
a processor coupled to the posture sensor, the processor being programmed to process the electrical signals from the posture sensor using hysteresis, and to estimate one of a plurality of posture states based on the processed electrical signals; and
a transceiver module programmed to transmit the estimate of the one posture state to an external device.

9. The device of claim 8, wherein the processor is programmed to define a transition band about a transition line between two posture states to implement hysteresis.

10. The device of claim 9, wherein the transition band includes first and second thresholds, wherein the first and second thresholds are predetermined.

11. The device of claim 9, wherein the transition band includes first and second thresholds, wherein the first and second thresholds are varied over time.

12. The device of claim 8, wherein the posture sensor is configured to sense the orientation in one dimension.

13. The device of claim 8, wherein the posture sensor is configured to sense the orientation in two or more dimensions.

14. The device of claim 8, further comprising a therapy module coupled to the processor, the therapy module being configured to deliver therapy.

15. A method for estimating posture using an implanted device, the method comprising:
generating one or more signals indicative of an orientation of the device relative to a direction of gravity;
processing the signals by defining a transition band about a transition line between posture states of a plurality of posture states; and
estimating one of the plurality of posture states based on the processed signals.

16. The method of claim 15, further comprising transmitting the estimate of the one posture state to an external device.

17. The method of claim 15, wherein processing the signals further comprises using hysteresis to process the signals.

18. The method of claim 15, wherein defining further comprises defining the transition band to include first and second thresholds, wherein the first and second thresholds are pre-determined.

19. The method of claim 15, wherein defining further comprises defining the transition band to include first and second thresholds, wherein the first and second thresholds are varied over time.

20. The method of claim 15, wherein estimating further comprises:
estimating a change in posture from a first posture state to a second posture state of the plurality of posture states when the signals go above a first threshold of the transition band; and
estimating a change in posture from the second posture state to the first posture state of the plurality of posture states when the signals go below a second threshold of the transition band.
